(19) United States
(12) Patent Application Publication
(10) Pub. No.: US 2014/0213912 A1
(43) Pub. Date: Jul. 31, 2014

(54) LOW POWER MONITORING SYSTEMS AND METHOD

(71) Applicant: COVIDIEN LP, Boulder, CO (US)

(72) Inventor: Mark Su, Boulder, CO (US)

(73) Assignee: COVIDIEN LP, Boulder, CO (US)

(21) Appl. No.: 13/753,007

(22) Filed: Jan. 29, 2013

(51) Int. Cl. A61B 5/00; A61B 5/02

(52) U.S. Cl. CPC A61B 5/0017; A61B 5/02
USPC 600/479; 600/476

(57) ABSTRACT

The present disclosure relates to systems and methods for collecting patient data via a monitoring system, with reduced power consumption. In one embodiment, the monitoring system is configured to emit pulses of light, and detect the light after passing through patient tissue. The light data is emitted sporadically, and the patient physiological data is reconstructed from the sporadically sampled light data. The sporadic sampling may reduce the power consumption by the monitoring system.

### BACKGROUND

The present disclosure relates generally to medical sensors and, more particularly, to low power medical sensors.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor and sense certain physiological characteristics of their patients. Accordingly, a wide variety of devices has been developed for monitoring and sensing many such physiological characteristics. For example, one category of monitoring and sensing devices includes spectrophotometric monitors and sensors. This category of device studies the electromagnetic spectra (e.g., wavelengths of light) and can monitor a suite of physiological parameters. Such devices provide doctors and other healthcare personnel with the information they need to provide quality healthcare for their patients. As a result, such monitoring and sensing devices have become an indispensable part of modern medicine.

Conventional spectrophotometric sensors are typically connected to a monitor via a cable. The cable provides the sensor with power and acts as a conduit for the transmission of signals between the sensor and the monitor. However, the cable also acts to tether the patient to the monitor, preventing unencumbered motion by the patient. As a result, such cable-based systems may not be suitable for ambulatory patients or for applications that require remote monitoring in non-clinical environments. Accordingly, various systems have been proposed which include a patient sensing device connected to a local monitor by way of a wireless link. Unfortunately, sensors that incorporate a wireless link may be limited to power provided on the sensor itself, which may be drained very quickly.

### BRIEF DESCRIPTION OF THE DRAWINGS

Advantages of the disclosed techniques may become apparent upon reading the following detailed description and upon reference to the drawings in which:

FIG. 1 illustrates components of a wireless medical sensor system, in accordance with one embodiment of the present disclosure;

FIG. 2 depicts a schematic diagram of the system of FIG. 1, in accordance with one embodiment of the present disclosure;

FIG. 3 depicts a flow diagram of a method for obtaining a physiological parameter from a patient, in accordance with an embodiment of the present disclosure;

FIG. 4 depicts a flow diagram of a method for calibrating the wireless medical system of FIG. 1, in accordance with an embodiment of the present disclosure;

FIG. 5 depicts a flow diagram of a method for obtaining an estimated physiological parameter from the wireless medical system of FIG. 1, in accordance with an embodiment of the present disclosure; and

FIG. 6 depicts a schematic diagram of the system of FIG. 1, in accordance with one embodiment of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions may be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Present embodiments relate to systems, methods, and devices for improving power consumption and lifespan of wireless medical sensors. Present embodiments may apply to a variety of wireless medical sensors, including sensors for measuring a photoplethysmograph, patient temperature, transvascular fluid exchange volumes, tissue hydration, blood flow, blood microcirculation, respiration, blood pressure, ECG (electrocardiography), pulse transit time, and/or others. For example, in one embodiment, a medical sensor system may include a medical sensor and a patient monitor that communicate with each other via wireless transmission circuitry. The sensor sporadically emits pulses of light (e.g., randomly or at predetermined irregular intervals) into a patient's tissue, in response to light drive signals. A detector, which may also be located on the sensor, detects the light attenuated by the patient's tissue, and collects data samples corresponding to the detected sporadic light pulses. Either the sensor or monitor may process the data samples to estimate physiological data and/or to generate a synthetic photoplethysmograph. The present disclosure provides systems and method for decreasing the amount of power used in collecting such data and measurements by configuring the sensor device to emit sporadic pulses of light rather than regular, frequent emission of light. This may reduce the amount of time that the sensor device spends in sensing the patient. The sensor device may expend less power and, accordingly, may have a longer battery life and overall lifespan.

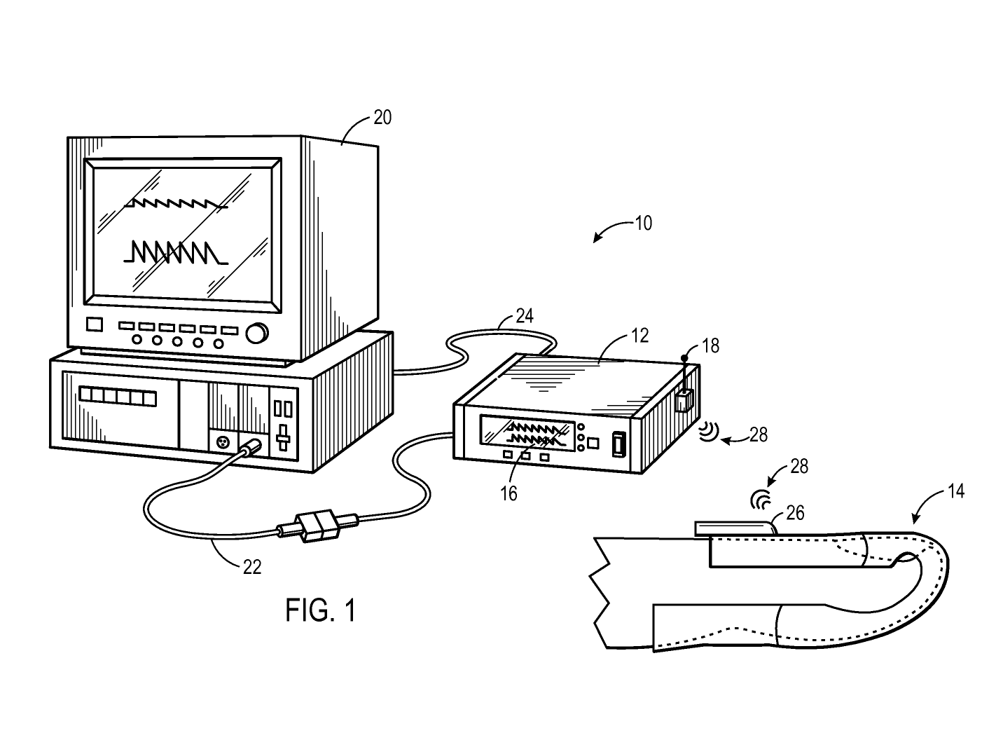

With the foregoing in mind, FIG. 1 depicts an embodiment of a wireless medical sensor system 10 that may efficiently sense physiological characteristics of a patient with sporadic light pulses, thereby conserving power. Although the embodiment of the system 10 illustrated in FIG. 1 relates to wireless photoplethysmography, the system 10 may be configured to obtain a variety of medical measurements with a suitable medical sensor. For example, the system 10 may, additionally or alternatively, be configured to measure patient temperature, transvascular fluid exchange volumes, tissue hydration, blood flow, blood microcirculation, respiration, ECG, non-invasive blood pressures (NIBP), blood pulse transit time, and/or others. In the illustrated embodiment, the system 10 includes a patient monitor 12 that communicates wirelessly with a sensor 14. In certain embodiments, the patient monitor 12 and the sensor 14 may communicate over a wired connection, such as through a cable connecting the sensor 14 to the monitor 12. The application of embodiments herein to reduce power consumption by the sensor may be useful for both wired and wireless systems.

The patient monitor 12 may include a display 16, a wireless module 18 for transmitting and receiving wireless data, a memory, a processor, and various monitoring and control features. Based on data received from the wireless sensor 14, the patient monitor 12 may display patient measurements and perform various measurement or processing algorithms. For example, when the system 10 is configured for pulse oximetry, the patient monitor 12 may perform blood oxygen saturation calculations, pulse measurements, and other measurements based on the data received from the wireless sensor 14. Furthermore, to provide additional functions, the patient monitor 12 may be coupled to a multi-parameter patient monitor 20, for example via a cable 22 connected to a sensor input port or via a cable 24 connected to a digital communication port. The multi-parameter patient module 20 may process and/or display physiological parameters from other sensors in addition to the data from the monitor 12 and sensor 14.

Like the patient monitor 12, the sensor 14 may include a wireless module 26. The wireless module 26 of the wireless sensor 14 may establish a wireless communication 28 with the wireless module 18 of the patient monitor 12 using any suitable protocol. By way of example, the wireless module 26 may be capable of communicating using the IEEE 802.15.4 standard, and may communicate, for example, using ZigBee, WirelessHART, or MiWi protocols. Additionally or alternatively, the wireless module 26 may be capable of communicating using the Bluetooth standard or one or more of the IEEE 802.11 standards. In an embodiment, the wireless module 26 includes a transmitter (such as an antenna) for transmitting wireless data, and the wireless module 18 includes a receiver (such as an antenna) for receiving wireless data. In an embodiment, the wireless module 26 also includes a receiver for receiving instructions (such as instructions to switch modes), and the wireless module 18 also includes a transmitter for sending instructions to the sensor.

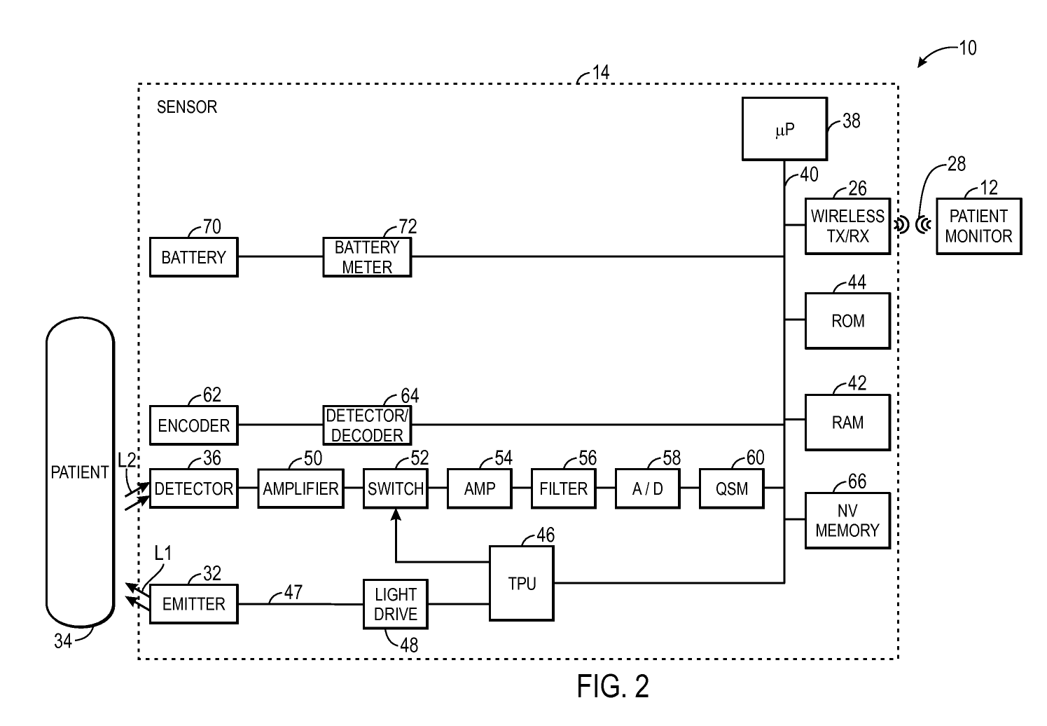

FIG. 2 is a block diagram of an embodiment of the wireless medical sensor system 10 that may be configured to implement the techniques described herein. By way of example, embodiments of the system 10 may be implemented with any suitable medical sensor and patient monitor, such as those available from Nellcor Puritan Bennett LLC. The system 10 may include the patient monitor 12 and the sensor 14, which may be configured to obtain, for example, a plethysmographic signal from patient tissue at certain wavelengths. The sensor 14 may be communicatively connected to the patient monitor 12 via wireless communication 28. When the system 10 is operating, light L1 from an emitter 32 (e.g., light at certain wavelength(s)) may pass into a patient 34 where the portions of the light may be differentially scattered, absorbed, and/or transmitted. Light L2 that emerges from the patient tissue may detected by a detector 36. For example, the system 10 may emit light L1 from two or more LEDs or other suitable light sources into a pulsatile tissue. The reflected or attenuated light L2 may be detected with the detector 30, such as a photodiode or photo-detector, after the light has passed through or has been reflected by the pulsatile tissue. In certain embodiments, the emitter 32 may be configured to emit pulses of light at random or preset irregular intervals.

The sensor 14 may include a microprocessor 38 connected to an internal bus 40. Also connected to the bus 40 may be a RAM memory 42 and a ROM memory 44. A time processing unit (TPU) 46 may provide timing control signals to a light drive circuitry 48 which may control when the emitter 32 is illuminated, and if multiple wavelengths are emitted, the multiplexed timing for the different wavelengths. The TPU 46 may also control the gating-in of signals from the detector 36 through an amplifier 50 and a switching circuit 52. These signals may be sampled at the proper time, depending upon which of multiple wavelengths of light is emitted, if multiple wavelengths are used. In one embodiment, the received signal from the detector 36 may be passed through an amplifier 54, a low pass filter 56, and/or an analog-to-digital converter 58.

The digital data may then be stored in a queued serial module (QSM) 60, for later downloading to the RAM 42 as the QSM 60 fills up. In one embodiment, there may be multiple parallel paths of separate amplifier, filter and/or A/D converters for multiple light wavelengths or spectra received. This raw digital data may be further processed by the circuitry of the wireless medical sensor 14 into specific data of interest, such as pulse rate (heart rate), blood oxygen saturation, and so forth. Alternatively, the raw digital data may be transmitted to the patient monitor 12, where it may be further processed into specific data of interest.

In an embodiment, the sensor 14 may also contain an encoder 62 that encodes information indicating the wavelength of one or more light sources of the emitter 32, which may allow for selection of appropriate calibration coefficients for calculating a physiological parameter such as blood oxygen saturation. The encoder 62 may, for instance, be a coded resistor, EEPROM or other coding devices (such as a capacitor, inductor, PROM, RFID, parallel resonant circuits, or a colorimetric indicator) that may provide a signal to the processor 38 related to the characteristics of the sensor 14 that may allow the processor 38 to determine the appropriate calibration characteristics for the photoplethysmographic components of the sensor 14. Further, the encoder 62 may include encryption coding that prevents a disposable or replaceable part of the sensor 14 from being recognized without corresponding adjustment or replacement of the information encoded by the encoder 62. In some embodiments, the encoder 62 and/or the detector/decoder 64 may not be present. Additionally or alternatively, the processor 38 may encode processed sensor data before transmission of the data to the patient monitor 12.

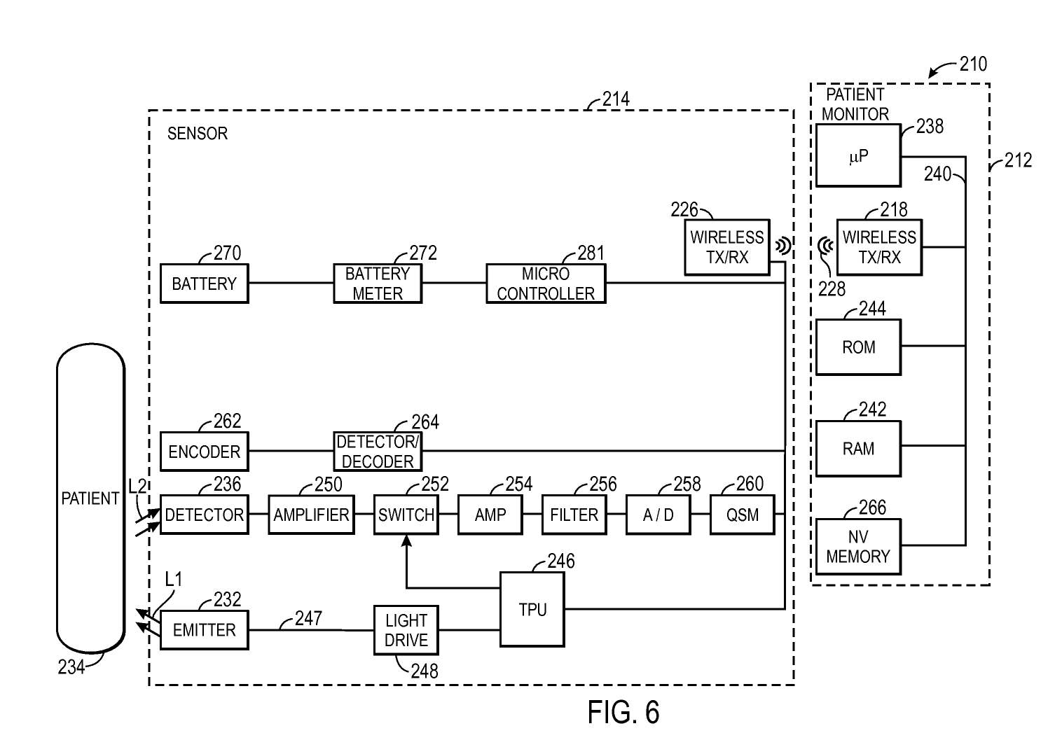

It should be noted that the patient monitor 12 may include the components described above for the sensor 14 with a few exceptions (e.g., emitter 32 and detector 36) to implement the techniques described herein. A wireless medical sensor system 210 including a sensor 214 and patient monitor 212 according to another embodiment is shown in FIG. 6. As shown in FIG. 6, the microprocessor 238, ROM 244, RAM 242, and NV Memory 266 are located on the patient monitor 212. In this case, the calculation of the physiological parameter by the processor 238 is accomplished on the monitor 212, rather than on the sensor 214. The sensor 214 operates similarly to that described above, by emitting light pulses into patient tissue, and detecting the reflected and/or scattered light. This raw light data is then passed to the microprocessor 238 on the monitor for further processing. The sensor 214 may also include a microcontroller 281, which controls the other components on the sensor.

Locating the microprocessor 238 and memory components 244, 242, 266 on the monitor 212 may reduce the power consumption and size of the wireless sensor 214. The sensor 214 acquires light data via the emitter 232 and detector 236, and transmits raw light data to the monitor 212 via wireless communication 228. The monitor 212 then processes the raw data to calculate the physiological parameter(s), as described in further detail below.

In an embodiment, the sensor 214 may transmit the acquired signal data to another type of device, in addition to or instead of the monitor 212. The other device may be, for example, a smart phone, a laptop computer, a remote computer, a handheld computing device, or a cloud computing device. In an embodiment, the sensor 214 transmits data to a wireless network, which may process and/or store the data via various networked processors or memory devices.

In various embodiments, based at least in part upon the value of the received signals corresponding to the light L2 received by detector 36, the microprocessor 38 (or the processor 238 on the monitor 12) may calculate a physiological parameter of interest using various algorithms. For example, the microprocessor 38 may utilize algorithms (e.g., analog-to-information sensing algorithm) to estimate a physiological parameter, morphological features, and/or a photoplethysmograph from sampled data acquired from the detected pulses of light. These algorithms may utilize coefficients, which may be empirically determined, corresponding to, for example, the wavelengths of light used. These may be stored in the ROM 44 or nonvolatile memory 66. In a two-wavelength system, the particular set of coefficients chosen for any pair of wavelength spectra may be determined by the value indicated by the encoder 62 corresponding to a particular light source of the emitter 32. For example, the first wavelength may be a wavelength that is highly sensitive to small quantities of deoxyhemoglobin in blood, and the second wavelength may be a complimentary wavelength. Specifically, for example, such wavelengths may be produced by orange, red, infrared, green, and/or yellow LEDs. Each wavelength may be associated with a different coefficient stored in the encoder 62. Different wavelengths may be selected based on instructions or protocols received from the patient monitor 12, based on preferences stored in a nonvolatile storage 66, or based on user input. User input may be inputted at the monitor 12, such as by a user interface provided on the monitor, and/or may be inputted to a remote host computer, which communicates with the monitor 12 via a suitable port or communication link. The instructions from the patient monitor 12 may be transmitted wirelessly to the sensor 14.

The nonvolatile memory 66 may store caregiver preferences, patient information, or various parameters, discussed below, which may be used in the operation of the sensor 14. Software for performing the configuration of the sensor 14 and for carrying out the techniques described herein may also be stored on the nonvolatile memory 66 and/or on the ROM 44. The nonvolatile memory 66 and/or RAM 42 may also store historical values of various discrete medical data points. By way of example, the nonvolatile memory 66 and/or RAM 42 may store the past or last known values for one or more physiological parameters such as oxygen saturation, pulse rate, respiratory rate, respiratory effort, blood pressure, vascular resistance, and/or vascular compliance. In an embodiment, the nonvolatile memory 66 and/or RAM 42 store the raw data points corresponding to the light L2 detected by the detector.

A battery 70 may supply the sensor 14 with operating power. By way of example, the battery 70 may be a rechargeable battery, such as a lithium ion or lithium polymer battery, or may be a single-use battery such as an alkaline or lithium battery. Due to the techniques described herein to reduce battery consumption, the battery 70 may be of a lower capacity, and accordingly smaller and/or cheaper, than a battery needed to power a similar sensor 14 that does not employ these techniques. A battery meter 72 may provide the expected remaining power of the battery 70 to a user and/or to the microprocessor 38.

The system 10 may be configured to sporadically emit pulses of light from the sensor 14 onto the patient at random or predetermined irregular (i.e., non-uniform) intervals, such that the emitter 32 is energized for a smaller amount of time than it would be conventionally. For example, pulses may be emitted at an average frequency in the range of every 100 to 700 milliseconds (ms), with a LED pulse length in the range of 10 to 50 ms. The average frequency is an average of the irregular intervals at which the pulses are emitted. As such, data collected by the detector 36 may be a set of sporadic data samples rather than a full data set (e.g., data gathered via frequent, regular emission of light for an extended length of time).

The light drive 48, 248 emits a light drive signal that instructs the emitter 32, 232 when to emit light. In an embodiment, the light drive signal includes a predetermined set of irregular intervals at which the emitter emits light. The light drive signal may include two such sets of irregular intervals, one for activating the red LED and a different one for activating the infrared LED, or the two LED's may be activated according to the same set of irregular intervals. The light drive signal may also be used to power the detector amplifier 50, 250 and/or ADC 58, 258 on and off, such that the amplifier and/or ADC is turned off between light pulses, to conserve power. Alternately, the detector and associated components may be left on continuously, to detect the light pulses L2 from the patient tissue any time they arrive at the detector.

The system 10 may use the sporadic data samples to estimate one or more physiological parameters of the patient, morphological parameters of the data samples, and/or a photoplethysmograph. These physiological parameters may include pulse rate, respiration rate, respiratory effort, blood pressure, vascular resistance, vascular compliance, oxygen saturation, and/or others. In some embodiments, these processes or acts may be done by a processor executing code in the sensor 14 or in the patient monitor 12.

In estimating a physiological parameter, signal probability distribution may be performed on the currently collected sporadic data samples (e.g., from detection of light L2, see FIG. 2, at one or more wavelengths such as red and infrared). Additionally, a Bayesian prior probability obtained from the last known measurement or value of the physiological parameter of the patient may be used along with the probability distribution of the current set of data samples to obtain a maximum likelihood frequency function for the physiological parameter. The maximum likelihood frequency function may be applied to the sporadic data samples to reconstruct a waveform representative of a photoplethysmograph (PPG) that fits the data samples. An embodiment of this process is illustrated in further detail below.

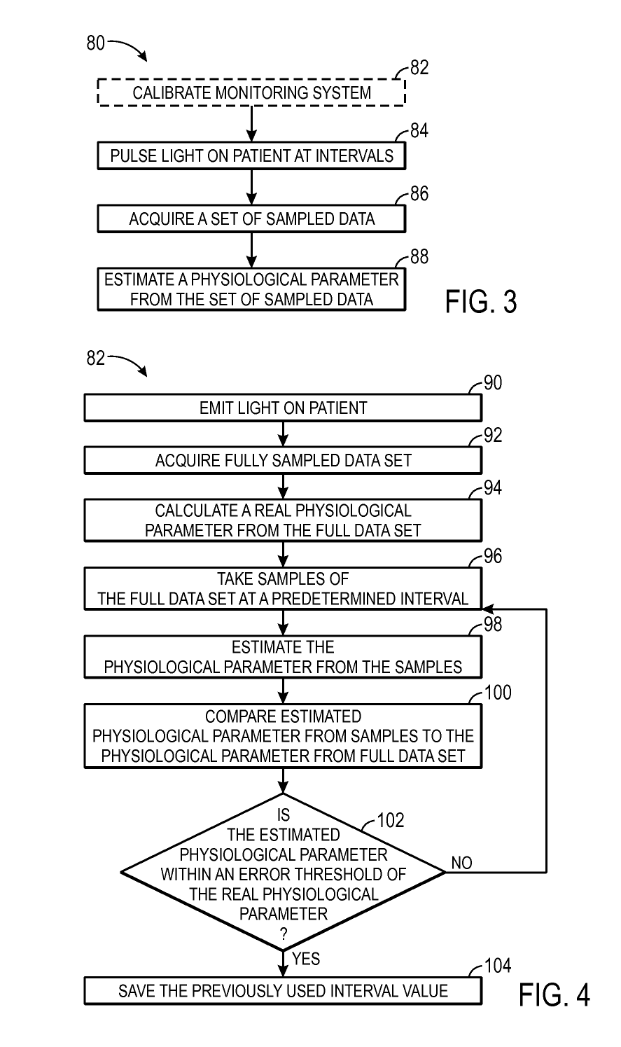

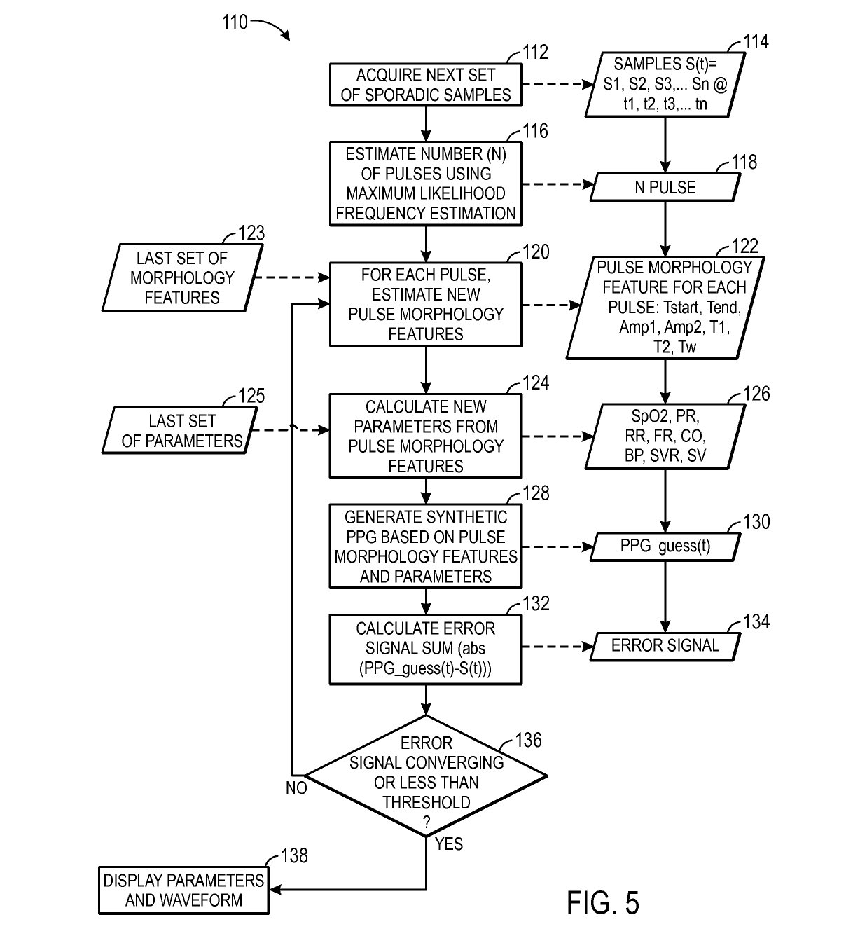

FIG. 3 illustrates a process 80 for obtaining a physiological parameter of the patient with the system 10, according to an embodiment of the present disclosure. In certain embodiments, the system 10 may first be calibrated (block 82) for the patient, as the system 10 may operate differently for different patients. However, in some embodiments, the system 10 may not be calibrated and thus, the calibration step (block 82) may be omitted. During the process, the system 10 may pulse (block 84) light on the patient via the emitter 32 of the sensor 14 at certain random or preset irregular intervals, for a certain duration. The emitter 32 may generally use power during the pulses and not use power or use very little power before, after, and/or in between the pulses. This reduces the power consumed by the emitter 32 during the process 80. The system 10 may then acquire (block 86) a set of sampled data representative of the detected pulses of light. This may be done by the detector 36 of the sensor 14. The system 10 may then estimate (block 88) a physiological parameter from the set of sampled data. In certain embodiments, the system 10 may estimate more than one physiological parameter. The calibration step (block 82) and the estimation step (block 88) will be shown in further detail in FIGS. 4 and 5, respectively.

As mentioned, in certain embodiments, the system 10 may be calibrated (block 82). Different patients may exhibit physiological parameters with varying degrees of stability. As such, the sampling frequency and duration to be used in pulsing light may be adjusted accordingly. For example, a patient with relatively stable physiological parameters may be subject to a lower sampling rate or duration as the sampled data may have a higher degree of accuracy. Conversely, a patient with relatively unstable physiological parameters may benefit from a higher sampling rate or duration in order to obtain sampled data with a sufficient degree of accuracy. Calibration may also be useful to account for variations in signal strength, patient perfusion, ambient noise, interference, and other factors. FIG. 4 illustrates the calibration process 82. During calibration, the system 10 may emit light (block 90) on to the patient (e.g., pulsatile tissue of the patient) for whom the system 10 is being calibrated.

To calibrate the system, the system first acquires a fully sampled data set (block 92) over a period of time. The fully sampled data set is obtained by emitting light (block 90) at regular intervals at a relatively high frequency, as compared to the sporadic pulses emitted at an overall lower average frequency during later operation. That is, the fully sampled data is obtained by emitting light at a higher frequency than the average frequency of the sporadic pulses of light. During this period of time, the emitter 32 emits light in regular pulses to acquire a fully sampled data set (block 92) rather than a sporadically sampled data set. The fully sampled data set may be a generally represent a complete measurement of the patient status (e.g., multiple physiological parameters), and may generally reflect the patient status with a high degree of accuracy. The system 10 may then calculate (block 94) a physiological parameter from the fully sampled data set. This may include converting the signals received from the detector 36 to a meaningful physiological parameter of the patient. Typically, physiological parameters obtained from the fully sampled data set have a high degree of accuracy.

After calculating the physiological parameter from the fully sampled data set (block 94), the system 10 then takes (block 96) a subset of sporadic samples from the fully sampled data set at pre-determined, irregular intervals. The result is a sub-set of sporadic data samples. Although the interval between samples varies in this sporadic data sub-set, the overall subset has an average sampling interval (e.g., average time between sampling, over the duration of the sampling), which is higher than the average sampling interval of the fully sampled data set (i.e., the average frequency is lower than the frequency of the fully sampled data set). This sub-sampling from the full data set may simulate a condition in which the system 10 pulses light at irregular intervals and collects the corresponding sampled data.

After taking the sporadic samples from the full data set, the system 10 may estimate a physiological parameter from the data subset (block 98). Other information, such as probability distributions and Base yean prior distributions may also be used in estimating the physiological parameter. The estimated physiological parameter may then be compared (block 100) to the physiological parameter calculated from the full data set (at previous block 94). The process 82 may then include determining if the estimated physiological parameter from the data subset is within a certain error threshold of the physiological parameter from the full data set (block 102). If the estimated physiological parameter from the data subset is indeed within (e.g., below) a certain error threshold of the physiological parameter calculated from the full data set, then the system 10 may save (block 104) the average interval value that was used to obtain the subset of samples (at block 96). This indicates that the sampling rate is sufficient and that the emitter may be configured to pulse light at such a rate. Otherwise, if the estimated physiological parameter from the data subset is not within a certain error threshold of the physiological parameter calculated from the full data set (e.g., equal to or above the error threshold), then the process 82 may include looping back to take another subset of samples of the full data set. This time, however, the average sampling interval used may be different than the interval previously used. Generally, the average sampling interval may be reduced such that more samples are taken. The process is then repeated to estimate a physiological parameter from the new data subset, and compare this value to the parameter calculated from the full data set, and the average sampling interval may be reduced as necessary to fall below the error threshold.

In certain embodiments, the process may also include a step in which the system 10 also detects if the estimated physiological parameter from the data subset is too close to the physiological parameter calculated from the full data set. This may indicate that the average sampling frequency is higher than it needs to be, and that a longer interval between sampling may be used to save power. Thus, in such an embodiment, the process 82 may include looping back to take samples of the full data set but increasing the average sampling interval instead. In other words, the system may operate with a maximum and minimum error threshold, and may calibrate via process 82 until the error threshold is between the maximum and minimum allowable amounts.

As mentioned, the system 10 is generally configured to pulse light sporadically (that is, at irregular or non-uniform intervals) on the patient in order to collect a set of sampled data (e.g., sporadically sampled data) representative of the patient status. The system 10 may then estimate one or more of a plurality of physiological parameters from the sampled data. FIG. 5 illustrates a detailed process 110 for obtaining one or more estimated physiological parameters. The process 110 starts by acquiring a set of sporadic samples 114 (block 112) from the light detector 36, 236 of the sensor 14, 214. The sporadic samples 114 may by expressed as a set, S(t), of sampled data values (S1, S2, S3, . . . Sₙ) and the corresponding times (t1, t2, t3, . . . tₙ) they were collected. The system 10 may then estimate (block 116) a number (N) of blood pulses (heart beats) from the sampled data values using maximum likelihood frequency estimation. The maximum likelihood frequency function may be generated from a signal probability distribution of the sampled data and a Bayesian prior probability based on the patient's last set of know physiological parameter values. Thus, the estimated physiological parameter 118 may be produced. In an embodiment, this estimation is performed by the processor 38, 238, on the sensor or the monitor, or other processing device.

In certain embodiments, the system 10 may estimate (block 120) a set of morphological features 122 from the estimated physiological parameter 118 and a last set of known morphological features 123 from the patient. In particular, for a photoplethysmograph or arterial pressure waveform the morphological features 122 for a individual cardiac pulse may be derived by fitting the pulse to two peaks and extracting the start and end times of the cardiac pulse (Tstart, Tend), the amplitude of the two fitted peaks (Amp1, Amp2), the time position of the two fitted peaks relative to Tstart (T1, and T2), and the width of the fitted peaks (Tw). This estimation (block 120) may be performed by the processor 38, 138.

In certain embodiments, the system 10 may calculate (block 124) new parameters 126 from the set of morphological features 122. The system 10 may utilize data from a last set of known physiological parameter values 125 from the patient in order to calculate the new physiological parameters 126. The physiological parameters 126 may include respiratory rate, pulse rate, respiratory effort, blood pressure, carbon dioxide levels, oxygen saturation, vascular resistance, vascular compliance, carbon monoxide level, stroke volume, and so forth. In an embodiment, this calculation (block 124) is performed by the processor 38, 138.

In certain embodiments, the system 10 may generate (block 128) a synthetic or reconstructed photoplethysmograph (PPG) 130 based on the set of morphological features 122 and the physiological parameters 118, 126. The generated PPG may be considered a guess or estimate (PPG_guess(t) 130). The system may then repeat the process through several iterations in order to obtain a PPG with an acceptable degree of accuracy.

In order to determine whether the photoplethysmograph 130 is within an acceptable degree of accuracy, the system 10 may calculate an error signal 134 (block 132). The error signal 134 is generally a difference between the photoplethysmograph 130 and the data samples 114 (the absolute value of PPG_guess(t) minus S(t)). In a sense, the system 10 is checking to determine if the reconstructed photoplethysmograph waveform 130 is similar to what the full data set would have provided. If the error signal 134 converges or is less than a threshold (block 136), then the system 10 may output or display (block 138) the physiological parameters and photoplethysmograph waveform 130. In certain embodiments, the threshold may be empirically derived.

The estimated parameters 118, 126 may be saved as the last set of parameters 125, and the estimated set of morphological features 122 may be saved as the last set of morphological features 123. If the error signal 134 does not converge or is not below the threshold (block 136) (that is, the error signal is equal to or above the threshold), then the system 10 may repeat the process. The system estimates (block 120) new sets of morphological features 122, calculates (block 124) new physiological parameters 126, generates (block 128) synthetic photoplethysmographs 130, and calculates (block 132) error signals 134 until the error signal 134 converges or is less than the threshold. In other words, the process 110 may include iteratively performing these acts until the error signal 134 converges or is less than the threshold.

The process outlined in FIG. 5 utilizes sporadic data sampling to calculate physiological parameters and generate a PPG waveform. These outputs are displayed (block 138) to the caregiver to indicate the patient's status. The parameters and PPG waveform are obtained at a desired level of accuracy from the sporadic data samples. By pulsing light at sporadic intervals, with an overall reduced sampling frequency, the system may reduce the overall power consumption of the sensor. The sensor operates to emit and detect light and provide detected light data to a processor, without operating at the full data sampling rate of conventional sensors. The reduction in light emission and data sampling can provide savings in power consumption, such that the wireless sensor can operate with a smaller battery and/or for a longer period of time between battery replacement or recharge.

Although various embodiments of a medical system and method have been disclosed herein, many modifications and variations will be apparent to those skilled in the art. It is to be understood that embodiments according to the present disclosure may be embodied other than as specifically described herein. The invention is also defined in the following claims.

What is claimed is:

1. A patient monitoring system for monitoring a physiological parameter of a patient, comprising:
a medical sensor, comprising:
an emitter configured to sporadically emit pulses of light; and
a detector configured to detect the sporadic pulses of light; and
a processor configured to receive sample data representative of the detected sporadic pulses of light from the medical sensor, wherein the processor is configured to execute code to estimate a value of at least one physiological parameter from the sample data.

2. The monitoring system of claim 1, wherein the medical sensor communicates with wirelessly with the processor.

3. The monitoring system of claim 1, wherein the at least one physiological parameter comprises pulse rate, respiratory rate, respiratory effort, blood pressure, vascular resistance, vascular compliance, carbon monoxide level, carbon dioxide level, stroke volume, or oxygen saturation.

4. The monitoring system of claim 1, wherein the processor is carried by the medical sensor.

5. The monitoring system of claim 1, further comprising a monitor comprising a display for displaying the at least one physiological parameter, and wherein the monitor comprises the processor.

6. The monitoring system of claim 1, wherein the processor is configured to execute code to determine a total number of blood pulses by generating maximum likelihood frequency data for the sample data.

7. The monitoring system of claim 6, wherein the processor is configured to estimate pulse morphological features of each pulse based on a last known set of morphological features for a last known value of the at least one physiological parameter.

8. The monitoring system of claim 7, wherein the processor is configured to execute code to estimate the value of the at least one physiological parameter based on the last known set of morphological features and the last known value.

9. The monitoring system of claim 1, wherein the sporadically emitted pulses of light comprise pulses of light emitted at random or pre-determined irregular intervals.

10. A method of monitoring a physiological parameter of a patient, comprising:
receiving a set of sporadic data samples, wherein the set of sporadic data samples was generated by sporadically emitting pulses of light on a patient and detecting the sporadic pulses of light scattered from the patient; and
estimating, using a processor, a value of at least one physiological parameter from the set of sporadic data samples by using at least one of a signal probability distribution of the set of sporadic data samples, maximum likelihood frequency data derived from the set of sporadic data samples, or a Bayesian prior probability of a last known value of the at least one physiological parameter.

11. The method of claim 10, further comprising generating a first set of morphological features for each blood pulse in the sporadic data samples, and wherein estimating the value of the at least one physiological parameter is based on at least the first set of morphological features.

12. The method of claim 11, further comprising generating a synthetic photoplethysmograph based on the first set of morphological features and the value of the at least one physiological parameter.

13. The method of claim 12, comprising:
generating an error signal by finding a difference between the synthetic photoplethysmograph and the set of sporadic data samples;
determining if the error signal is less than a threshold;
outputting at least one of the first set of morphological features, the at least one physiological parameter, or the synthetic photoplethysmograph if the error signal is determined to be less than the threshold; and
estimating a second set of morphological features if the error signal is determined to be equal to or above the threshold.

14. The method of claim 10, wherein the at least one physiological parameter comprises pulse rate, respiratory rate, respiratory effort, blood pressure, vascular resistance, vascular compliance, carbon monoxide level, carbon dioxide level, stroke volume, or oxygen saturation.

15. A method of obtaining physiological patient data comprising:
sporadically emitting pulses of light on a patient via an emitter of a medical sensor, the sporadic pulses of light having a first average frequency;
acquiring sampled data based on detected light scattered from the patient in response to the sporadic pulses of light; and
estimating, via a processor, at least one physiological parameter, a first set of morphological features, or a photoplethysmograph from the sampled data.

16. The method of claim 15, further comprising:
calibrating a monitoring system, wherein calibrating the monitoring system comprises:
emitting light on the patient for a duration of time, via light pulsed at regular intervals at a second average frequency higher than the first average frequency of the sporadic pulses;
acquiring a fully sampled data set based on the detected light scattered from the patient in response to the light emitted at the second average frequency;
sampling the fully sampled data set at an average sampling frequency to produce a data sub-set;
comparing a characteristic of the data sub-set to the fully sampled data set;
adjusting the average sampling frequency based on the comparison; and
sporadically pulsing light at the adjusted average sampling frequency.

17. The method of claim 16, wherein comparing the characteristic of the data sub-set to the fully sampled data set comprises:
calculating a first value for the at least one physiological parameter from the fully sampled data set;
estimating a second value for the at least one physiological parameter from the data sub-set;
determining if the second value is within an error threshold of the first value.

18. The method of claim 17, wherein adjusting the average sampling frequency based on the comparison comprises:
increasing or decreasing the average sampling frequency, sampling the fully sampled data set at the increased or decreased average sampling frequency to produce a second data sub-set, and comparing the characteristic of the second data sub-set to the fully sampled data set if the second value is determined to not be within the error threshold of the first value; and
saving the average sampling frequency if the second value is determined to be within the error threshold of the first value.

19. The method of claim 15, comprising outputting at least one of a number of blood pulses, the first set of morphological features, the at least one physiological parameter, or the photoplethysmograph.

20. The method of claim 15, wherein the at least one physiological parameter comprises at least one of pulse rate, respiratory rate, respiratory effort, blood pressure, vascular resistance, vascular compliance, carbon monoxide level, carbon dioxide level, stroke volume, or oxygen saturation.
